(19) United States
(12) Patent Application Publication
(10) Pub. No.: US 2005/0049517 A1
(43) Pub. Date: Mar. 3, 2005

(54) Electromyogram method and apparatus

(75) Inventors: Thomas Mathew, Skokie, IL (US); Di-An Hong, Inverness, IL (US); George Valliath, Buffalo Grove, IL (US); William L. Olson, Lake Villa, IL (US)

Correspondence Address: FITCH EVEN TABIN AND FLANNERY, 120 SOUTH LA SALLE STREET, SUITE 1600, CHICAGO, IL, 60603-3406, US

(73) Assignee: Motorola, Inc.

(21) Appl. No.: 10/654,495

(22) Filed: Sep. 3, 2003

(51) Int. Cl. A61B 5/04

(52) U.S. Cl. 600/546

(57) ABSTRACT

An electromyogram device (10) have a conformable housing (11) that houses or otherwise supports an electromyogram signal processor (13), electromyogram sensors (14), and a display (15) that provides, for example, information corresponding to one or more muscle condition parameters such as muscular fatigue. In various embodiments, the device can further include additional displays (34), memory (31), audible alarm mechanisms (32), and/or a wireless receiver and/or transmitter (33). In one embodiment, such devices can communicate amongst one another to permit central and/or remote display of the resultant electromyogram information.

### TECHNICAL FIELD

This invention relates generally to electromyogram sensing.

### BACKGROUND

Muscle tissue contracts and relaxes as a function, at least in part, of the presence and absence of triggering biologically-based electrical signals. Sensors can be employed to detect such signals. The resultant waveforms are typically referred to as electromyograms.

Such sensors must ordinarily be located in direct contact with a skin surface proximal to the muscle tissue of interest. Corresponding electrodes are utilized to source a small potential across a portion of the muscle tissue and another electrode serves to detect the electrical response of the muscle tissue to this potential.

The resultant electromyogram information can be helpful to diagnose various medical conditions having characteristic symptomatic muscle tissue conditions. Such information could also potentially be used with respect to various physical activities (such as sports training or physical rehabilitation) that have a corresponding characteristic desired or expected muscle tissue reaction. To date, however, employment of such information remains relatively restricted. This likely results, at least in part, due to relatively significant barriers to obtaining and then rendering useful such information.

For example, obtaining electromyograms typically entails deployment and subsequent equipment operation by a skilled and trained operator. Appropriate placement of the electrodes with respect to one another, for example, typically requires a priori experience and training regarding such systems. Skill and expertise regarding affixing the electrodes in a desired position using, for example, tape or other adhesives can also present an obstacle to usage by ordinary people. Furthermore, electromyogram information itself yields relatively non-intuitive results to all but trained and experienced interpreters.

As a result, obtainment and use of electromyogram information remains largely confined to relatively limited clinic applications.

### BRIEF DESCRIPTION OF THE DRAWINGS

The above needs are at least partially met through provision of the electromyogram method and apparatus described in the following detailed description, particularly when studied in conjunction with the drawings, wherein:

FIG. 1 comprises a top plan schematic depiction as configured in accordance with an embodiment of the invention;

FIG. 2 comprises a top plan view of a display as configured in accordance with an embodiment of the invention;

FIG. 3 comprises a block diagram as configured in accordance with an embodiment of the invention;

FIG. 4 comprises a detail block diagram as configured in accordance with various embodiments of the invention;

FIG. 5 comprises a front elevational schematic depiction as configured in accordance with various embodiments of the invention;

FIG. 6 comprises a flow diagram as configured in accordance with various embodiments of the invention;

FIG. 7 comprises a detail flow diagram as configured in accordance with various embodiments of the invention; and

FIG. 8 comprises a flow diagram as configured in accordance with an embodiment of the invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are typically not depicted in order to facilitate a less obstructed view of these various embodiments of the present invention.

### DETAILED DESCRIPTION

Generally speaking, pursuant to these various embodiments, a conformable housing supports an electromyogram sensor, an electromyogram signal processor, and a display. In a preferred embodiment, the conformable housing is of sufficient size to permit substantially conformal disposition about a human body part (such as, but not limited to, a portion of an arm or of a leg). In a preferred approach, the electromyogram sensor joins the conformable housing in such as fashion as to permit the sensor to detect muscle activity when the conformable housing is substantially conformally disposed about a human body part. Pursuant to a variety of embodiments, the display provides information (in textual, graphical, or other suitable form) that corresponds to a muscle condition parameter (such as, but not limited to, muscular fatigue). Such information can be, if desired, a composite indicia that corresponds to a condition of, for example, a plurality of muscles or a particular muscle as viewed, for example, over time.

In some embodiments, a memory also couples to the electromyogram signal processor. This memory can provide information that the electromyogram signal processor utilizes when processing the electromyogram sensor data. As one example, the memory can include comparative (and/or calibration) information regarding strength (for example, for a given specific individual, as corresponds in general to a particular target demographic audience, or otherwise). As another example, the memory can include information regarding past performances (such as, for example, a past number or rate of muscle flextures or muscle relaxation events for a given individual).

Pursuant to one embodiment, a plurality of distally positioned electromyogram sensor mechanisms can provide information to a given electromyogram signal processor to permit a unified or discrete presentation of corresponding muscle status information. When using a plurality of sensors, the resultant information can be provided to the electromyogram signal processor via a wireline and/or a wireless channel. If desired, the electromyogram signal processor itself can also be provided with a transmitter capability. So configured, the electromyogram signal processor can, for example, provide processed muscle activity or status information to a remote display and/or to another processing unit as desired.

In some applications it may be appropriate to provide a calibration capability. So configured, the resultant apparatus can be calibrated with respect to a given individual (or, for example, a class of individuals) to facilitate a more accurate interpretation of the resultant electromyogram waveforms. Such calibration can make use of locally stored calibration and/or remotely stored calibration information as may be appropriate to the given application. Such calibration can also be based upon specific actions taken by a user in response, for example, to specific calibration instructions as provided to the user.

So configured, a relatively untrained individual can make beneficial and accurate use of electromyogram waveform information. For example, these embodiments tend to permit proper disposition and use of the electromyogram sensors without requiring a copious amount of training regarding proper placement of the electrodes (particularly with respect to one another). These embodiments also tend to facilitate relatively rapid placement and removal of the electromyogram sensors as, essentially, a beneficial side effect of simply donning the conformable housing. These embodiments also support the provision of relatively simple and intuitive information that is nevertheless of value and benefit to an untrained and unskilled observer. This, in turn, permits use of such embodiments in a wide variety of non-clinical settings and for a wide variety of non-traditional purposes. For example, such electromyogram information can be used to supplement or even define a wide variety of physical training activities, including training for competitive sports as well as physical therapies. Furthermore, such benefits are attainable at a relatively modest cost.

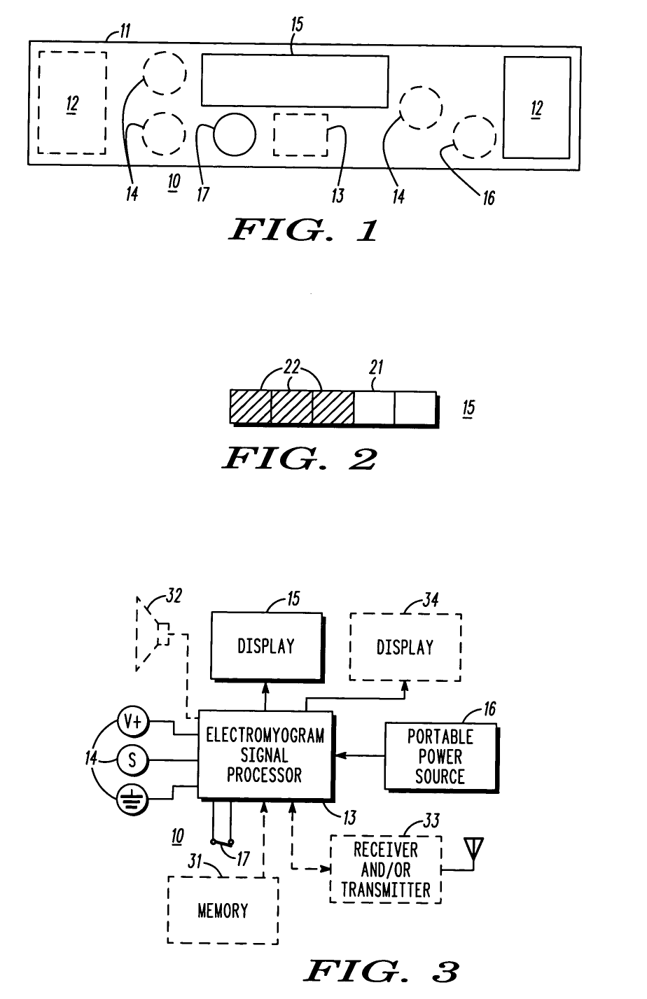

Referring now to the drawings, and in particular to FIG. 1, an electromyogram device 10 comprises, in a preferred embodiment, a conformable housing 11 comprised of, for example, an elastic material or an otherwise pliable material such as cloth or certain plastics. The conformable housing 11 preferably includes some mechanism to facilitate retention of the conformable housing once properly positioned on a body part. For example, hooks and loops 12 can be utilized to permit such retention in a manner well understood in the art. Other mechanisms could be used as well, including snaps, zippers, ties, magnets, and so forth.

It is possible that the conformable housing 11 could be offered in various lengths and widths to facilitate accommodation of a wider variety of body part sizes and shapes. For example, a longer conformable housing might be preferred for use with the upper torso while a shorter conformable housing may be better suited to use with a smaller body part (such as a bicep muscle area of the arm). Depending upon the choice of materials, the conformable housing 11 can be comprised of a single layer (having cavities or other support mechanisms incorporated therein to permit support of the other elements described below) or can by comprised of multiple layers as desired. When using multiple layers, one may also elect to utilize layers of differing materials to better accommodate some particular design requirement (regarding, for example, weight, water resistance, strength, washability, breathability, printability, and so forth).

The conformable housing 11 serves as a housing and/or as a support substrate for a number of other preferred and/or optional components. In a preferred embodiment, these include an electromyogram signal processor 13, an electromyogram sensor (comprised in this depiction of three electrodes 14 as comports with ordinary prior art practice), and a display 15. The electromyogram signal processor 13 can be architecturally comprised of a single integrated device or a plurality of devices as desired and/or appropriate to a given application. In addition, the electromyogram signal processor 13 can comprise a fixed-purpose platform or can be at least partially programmable. Such architectural variations and options are generally well understood in the art. In general, the electromyogram signal processor 13 will preferably be relatively small (such as a single integrated circuit) and consume only a small amount of power. This signal processor 13 serves, in general, to receive the electromyogram signals from the electromyogram sensor and to process those signals to at least provide corresponding information via the display 15. (Additional description regarding operation of the electromyogram signal processor 13 appears below where appropriate.)

The display 15 can be any of a wide variety of display technologies including, but not limited to, a liquid crystal display. In a preferred embodiment the display 15 comprises a multi-color display and may optionally comprise an alphanumeric display to permit provision of textual content. In general, this display 15 serves to at least provide information that corresponds to a muscle condition parameter. For example, the display 15 can provide indicia that corresponds to muscular fatigue. As one exemplary illustration of this capability, and referring momentarily to FIG. 2, the display 15 can be comprised of a plurality of segments 21. These segments can be serially and contiguously illuminated in correspondence to muscular fatigue (wherein the electromyogram signal processor 13 determines the degree of fatigue as a function, at least in part, of electromyogram signals from the electromyogram sensor). For example, three of the segments 22 can be illuminated to indicate a present degree of muscular fatigue that falls within a corresponding range of relative fatigue. It will be appreciated that such a display provides a relatively intuitive indication and measure of such a muscle condition parameter and therefore can be relatively quickly understood by many or most individuals and acted upon accordingly.

In this embodiment, the conformable housing 11 also supports a portable power source 16. The portable power source 16 can comprise, for example, one or more batteries. Such batteries can be disposed on an outer surface of the conformable housing 11 or can be disposed within the housing 11. The portable power source 16 can be disposed within a pocket or other opening in the conformable housing 11 to permit access thereto. Such access will facilitate servicing and changing the portable power source 16. In the alternative, if desired, the portable power supply 16 can be more permanently secured within the conformable housing 11. Such disposition may be appropriate when using, for example, a rechargeable portable power source. Charging electrodes can be externally provided when using a rechargeable portable power source. In the alternative, the rechargeable portable power source can comprise a non-contact rechargeable portable power source as is known in the art. (Such non-contact rechargeable portable power sources use, for example, inductive coupling to facilitate recharging the power source.)

In an optional embodiment, the conformable housing 11 can also support a user input interface 17. This user input interface 17 may comprise, for example, a switch such as a push button switch. Other input mechanisms, including multi-switch mechanisms, are of course possible to suit the needs of a given application. Such a user input interface 17 can be used to accommodate a variety of purposes. For example, a user can use such an interface 17 to place the electromyogram signal processor into an active mode of operation. Other uses are also possible.

The electromyogram device 10 can include other elements and features as well. At least some other possibilities are noted below where appropriate.

So configured, the electromyogram device 10 can be readily disposed about a muscle of interest. Notwithstanding placement by a person with little or no electromyogram sensor training, the described form factor will nevertheless tend to encourage proper placement of the various electrodes 14. These embodiments also permit relatively rapid placement of the device 10 and removal as well. Neither the individual components themselves nor the manner of their combination requires extraordinary skill, equipment, or expense to successfully employ.

Referring now to FIG. 3, the electromyogram device 10 can be functionally configured as illustrated. In a preferred embodiment, the electromyogram signal processor 13 operably couples to the three electromyogram electrodes 14 (comprising, in accordance with known practice, V+ and ground electrodes across which a stimulating potential is applied and a sense (S) electrode to facilitate sensing the electrical status (and corresponding response) of the proximal muscle tissue. The electromyogram signal processor 13 also operably couples to a display 15 as described above. If desired, the electromyogram signal processor 13 can optionally couple to one or more additional displays 34. Supplemental displays may be appropriate to accommodate particular form factor or other ergonomic requirements. Multiple displays may also be useful to facilitate display of other information (or of electromyogram information from remote sources pursuant to an optional approach described below).

If desired, the electromyogram signal processor 13 can also couple to one or more external memories 31. Such memory 31 can store electromyogram information as detected and/or processed by the electromyogram signal processor 13. Such memory can also hold other kinds of information, including but not limited to:
strength information as pertains to one or more individuals;
power information as pertains to one or more individuals;
a number of muscle flextures as occurs during some measurement period or periods;
a present rate of effecting flexures of a monitored muscle;
historical information regarding a previous rate of effecting muscle flextures;
a number of muscle relaxation events as occurred during some measurement period or periods;
a present rate of effecting muscle relaxation events for a monitored muscle;
historical information regarding a previous rate of effecting muscle relaxation events for a monitored muscle; and so forth. Other information, to support the processing of electromyogram information or otherwise, can also be stored in the memory 31 as may be desired and/or appropriate for a given application.

In one embodiment, the electromyogram device 10 can also include an audible alarm 32 that operably couples to, for example, the electromyogram signal processor 13. If desired or necessary, a power amplifier (not shown) can also be included to effect provision of an audible signal of desired amplitude. Such an audible alarm 32 can serve, for example, to signal attainment of a given state of being with respect to one or more monitored muscle condition parameters. To illustrate, an audible alarm can be sounded when a predetermined level of muscle fatigue occurs or when at least a given level of muscle fatigue persists for more than a predetermined period of time.

In another embodiment, the electromyogram device 10 can also include a wireless receiver and/or transmitter 33. Such a capability can serve a variety of purposes. For example, so configured, the electromyogram signal processor 13 can receive electromyogram signals from additional remote electromyogram sensors. This, in turn, will permit the electromyogram signal processor 13 to process (and compare and contrast as appropriate) signals that represent a current condition of more than a single muscle. A transmission capability will permit the electromyogram signal processor 13 to transmit, for example, electromyogram information (including, for example, display information) to, for example, a remote or supplemental display.

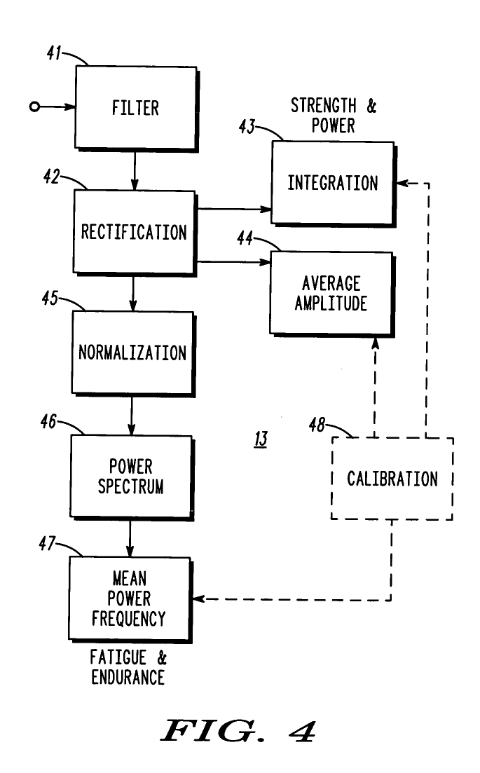

Referring now to FIG. 4, the electromyogram signal processor 13 can be configured as desired to process the electromyogram signals as provided by the electromyogram sensor. In general, the electromyogram signal processor 13 will typically filter 41 the incoming electromyogram signals and subject those filtered signals to rectification 42. In a preferred embodiment, this rectification 42 will typically comprise half-wave rectification. The resultant rectified electromyogram signal can then be processed in a variety of ways.

Pursuant to one approach, the rectified electromyogram signal is normalized 45 and then processed with respect to its power spectrum 46. The power for each frequency component represented by a corresponding Fast Fourier Transform can be obtained by squaring the magnitude of that frequency component; the “power spectrum” then relates to a plot of that power in each of the frequency components. The mean power frequency 47 is then ascertained to thereby provide information that corresponds to muscle fatigue and endurance. In a preferred approach, mean power frequency comprises a weighted average frequency in which each frequency component f is weighted by its power P (for example, P₁ is the power of f₁). More particularly, the mean power frequency can be obtained by summing the frequency times power of the components and then dividing by the sum of the powers. That is:
Mean power frequency=(f₁*P₁+f₂*P₂+. . . +fₙ*Pₙ)/(P₁+P₂+. . . +Pₙ)

Pursuant to another embodiment, the rectified electromyogram signal is integrated 43 to thereby provide information that corresponds to strength and power. Pursuant to yet another embodiment, the rectified electromyogram signal is subjected to an average amplitude 44 process to yield information that corresponds to muscle fatigue. As to the latter, the electromyogram signal amplitude increases as the simultaneous muscle action potentials sum. The average amplitude then comprises a calculation result for the sum of the electromyogram signal values over a designated interval divided by the time interval. Such average amplitude is often expressed as a percentage of the value with respect to so-called maximum voluntary contraction.

As muscle fatigue has been shown to be accompanied by increases in electromyogram signal amplitude and decreases in mean power frequency, such values can be helpful in interpreting the data obtained through these embodiments. For example, in order to calibrate a biceps fatigue level, a subject can perform repetitive elbow flexion-extension movements while holding a five kg weight in hand until volitional exhaustion occurs. Biceps brachii electromyogram signals can be recorded continuously during such a test and the corresponding electromyogram signal power spectrum and average amplitude then calculated accordingly. The mean power frequency and/or average amplitude can then be used to characterize the temporal history of changes and to express or characterize the electromyogram signal features at different fatigue levels.

The above approaches to developing information regarding fatigue, endurance, strength, and power are each understood in the art. Therefore, additional detail will not be provided here for the sake of brevity and the preservation of focus.

Any of the above described indicia can then be utilized by the electromyogram signal processor to develop a corresponding information display.

Pursuant to one approach, the electromyogram signal processor can display a numerical or alphanumeric value or representation that correlates to the absolute or relative value of the electromyogram information. Pursuant to another approach, the electromyogram signal processor can compare the electromyogram information against other information or thresholds to facilitate the display of a corresponding representation. For example, the electromyogram signal processor 13 can provide one informational presentation when the present monitored muscle condition parameter is less than a predetermined limit and another information presentation when the present monitored muscle condition parameter exceeds this predetermined limit.

As a more specific example, the electromyogram signal processor 13 can utilize the average amplitude 44 approach to extract information from the electromyogram signals regarding muscular fatigue. This extracted information can then be compared against a predetermined threshold that represents (for a given individual, a class of individuals, or such other point of comparison as may be useful and pertinent in a given setting), for example, a target level of activity or a safety limit beyond which the monitored individual should not exceed.

It would also be possible to use more than one of the above described methods (or, indeed, to use other methods now known or hereafter developed) in combination with one another to yield a more complete, accurate, and/or fusion-based informational result. For example, both mean power frequency 47 and average amplitude 44 can be utilized to develop a redundant and/or averaged view of muscular fatigue. As another example, both mean power frequency 47 and integration 43 can be utilized to matrix and/or fuse, as desired, metrics regarding both endurance and power to provide a composite vector regarding present muscular conditions. And, as before, the electromyogram signal processor 13 can then depict in any appropriate fashion the resultant information in a scalar and/or un-scaled relativistic fashion as desired.

It would be possible to effect the above processing in a manner that treats all monitored individuals as being essentially equal. For many purposes, however, it may be more appropriate to adjust the processing to reflect special conditions, needs, purposes, or aspirations of the particular individual being monitored. One way to accommodate such flexibility is to provide calibration 48 that effects appropriate corresponding changes in how the electromyogram signal processor 13 conducts the above described processing. Additional detail will be provided below regarding such calibration.

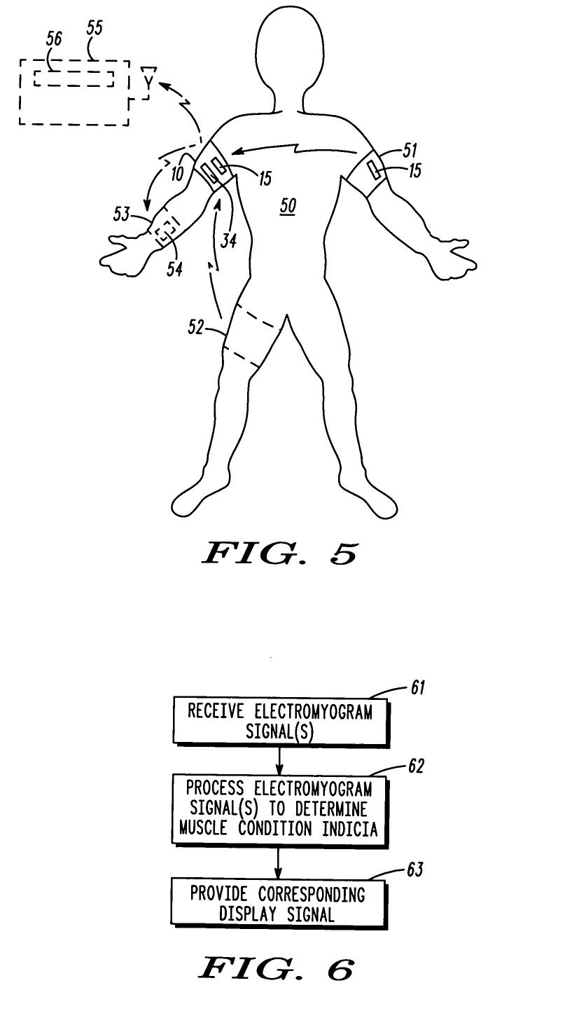

First, however, it may be helpful to first provide additional explanation with respect to various embodiments and configurations of usage that may be accommodated via some or all of these embodiments. With reference to FIG. 5, the electromyogram device 10 is readily disposed as described earlier about, for example, the arm of an individual 50. So positioned, the electromyogram device 10 will monitor the condition of the proximal muscle tissue with the corresponding resultant electromyogram information being displayed on the display 15.

As noted earlier, the device 10 can include a wireless receiver. Therefore, if desired, a second electromyogram device 51 can be disposed elsewhere on the individual 50 (for example, as illustrated, on the opposing arm). This second electromyogram device 51, when equipped with a transmitter as described above, can transmit its electromyogram information (either the raw data itself or the processed or partially processed data as appropriate to a given implementation) to the first electromyogram device 10. The first electromyogram device 10 can then utilize this additional information in a variety of ways. As one example, the additional information can be discretely displayed on a second display 34 as described above. As another example, the additional information can be fused with the locally developed information (for example, by averaging the additional information in a weighted or unweighted fashion with the locally developed information) to permit provision of a single displayed metric that represents a combined view of both muscle tissues. As yet another example, a single display 15 can be alternatively toggled between informational displays for both information sources. Other possibilities also exist.

Along these same lines, a secondary (or other supplemental) electromyogram device 52 that lacks a local display can also be used as desired. Such a device 52 may be particularly appropriate for use with muscle tissues where direct convenient observation of the display may not be readily possible. In such a case, and by providing the secondary electromyogram device 52 with a transmitter, the electromyogram information can be provided wirelessly to the first electromyogram device 10 where the remotely developed information can be displayed and/or otherwise processed as desired.

It would also be possible to provide a wearable mechanism 53 that comprises a remote display 54 and an appropriate receiver. So configured, the latter can be worn in a location where the individual 50 has convenient viewing access to the remote display 54 such that electromyogram information as developed by the electromyogram device 10 (either alone or as based upon inputs from other electromyogram devices) is displayed in a convenient location as selected by the individual 50. In a somewhat similar fashion the electromyogram device 10 can also transmit information to a remotely positioned receiver 55. For example, the electromyogram device 10 can transmit electromyogram information to be displayed on a display 56 upon reception by the receiver 55. Such a remote display mechanism can serve a variety of purposes. For example, the individual 50 could place the device in a convenient location distal to their body to possibly even more easily facilitate viewing the display. As another example, another individual, such as a monitoring trainer or physical therapist, could utilize such a remote display to remain conveniently apprised of the individual's muscular status and condition.

These or other suitable platforms can be used to effect a process such as that set forth in FIG. 6. Pursuant to this process, one receives 61 one or more electromyogram signals and processes 62 those signals to determine a corresponding muscle condition indicia. One then provides 63 a display signal (or signals) that corresponds to the muscle condition indicia. As already noted above, such electromyogram signals can be received from a local electromyogram sensor as may share a common housing or support surface with the processing platform itself and/or from remotely located electromyogram sensors. Such an electromyogram can be received via any appropriate signal conveyance pathway including electrically conductive channels, wireless channels (including, for example, radio frequency and infrared channels), and optical conduits such as optical fiber pathways and other optical waveguides and any combination thereof).

As noted above, the processing activity can serve to ascertain and quantify a variety of indicia regarding the monitored muscle tissue including, for example, muscle fatigue. It is also possible to process information concerning a plurality of muscles in order to determine, for example, a composite indicia that corresponds to the condition of such muscles. Such processing can comprise an effective measurement of the parameter or parameters of interest and/or the comparison of such parameters against one or more criteria. Such criteria can include, for example, one or more threshold values that correspond, for example, to a user performance parameter of interest. Such thresholds can be relatively static or dynamically determined and can be relatively broad-based or specific and personal to a given individual (for example, a given threshold value can be determined as a function, at least in part, of one or more past performances by the user).

As also noted above, such processing activity may also be based upon or otherwise utilize calibration information and/or a calibration process. There are various ways to effect or otherwise support such calibration activity. For example, and referring now to FIG. 7, a calibration process 70 can include an interactive calibration process 71 to specifically elicit the development of calibrating information in conjunction with the user. In one preferred approach the interactive calibration process 71 provides 72 a calibration instruction to a user using, for example, the display. Example calibration instructions are:
Flex the muscle five times;
Flex the muscle ten times at equal intervals within one minute;
Flex the muscle until flexing cannot be done without some discomfort; and so forth. Such examples are of course to be taken as illustrative in nature only and not as an exhaustive listing of possibly useful instructions.

If the user does not respond accordingly 73 (within, for example, a predetermined period of time T 74) to the calibration instruction, a predetermined action 75 can be taken. For example, the calibration instruction can again be provided to the user. As another example, the calibration instruction can be repeated in conjunction with provision of an audible alarm. These examples are illustrative of the concept and many other alternative actions are of course possible.

When the user does respond according to the calibration instruction, the calibration process 70 can then utilize the resultant electromyogram information when providing 77 the calibration criterion to be used during the above described processes. For example, when the calibration instruction requires the individual to effect flexures until noticeable fatigue becomes apparent, then the number of flextures as occurred during the calibration window (as can be determined automatically by the embodiments set forth above), plus or minus some constant or other weighting factor as may be appropriate to a given application, can be used when developing a threshold value to use when subsequently evaluating muscle activity for this individual (or other individuals of like circumstance when so desired).

In addition to such specifically developed user performance information, or in lieu thereof, the calibration process 70 can optionally comprise accessing 75 previously stored calibration information. Such accessing can comprise accessing locally stored calibration information and/or remotely stored calibration information (the latter via an appropriate communication link as comports with the resources and capabilities of a given setting). Such calibration information can comprise a wide variety of content. For example, information regarding past performance milestones of relevance for this particular individual can be accessed. As another example, similar information as corresponds to a group of people who are sufficiently similar to the present user to warrant such usage can be accessed. As yet another example, information regarding performance thresholds of interest for model performance can be accessed. This latter example would permit a given individual to compare their own capacity and performance against, for example, the performance of a role model, a composite standard, or a target level of performance, to name a few.

Pursuant to the various embodiments set forth above, muscles are readily monitored by an easily donned and readily understood electromyogram device. The resultant information can be used in various ways. For example, an individual may be able to effect a training regimin while assuming a reduced risk of injury through over exertion. As another example, a trainer may be able to better supervise the performance of a group of individuals. As yet another example, an individual may be able to more readily compare and contrast their present performance against their own past performance, target levels of performance, the performance of a role model, or some other relevant standard.

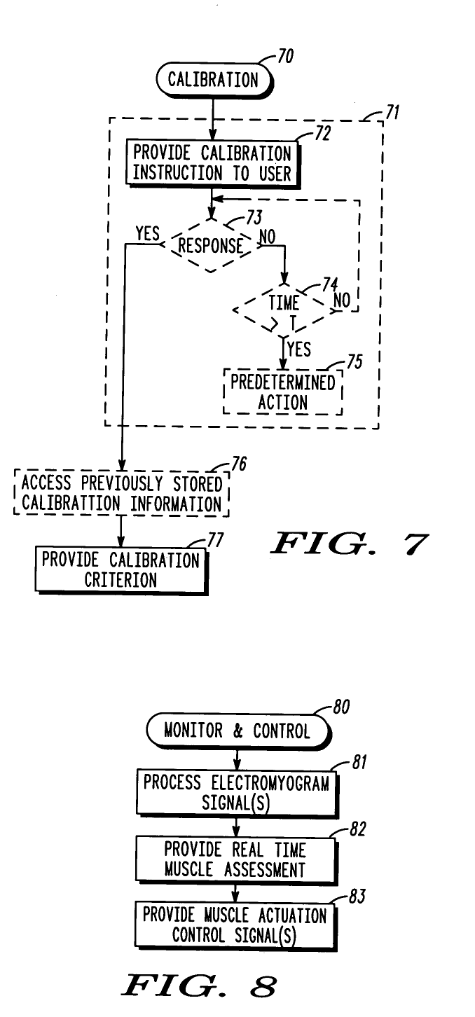

Those skilled in the art will recognize that a wide variety of modifications, alterations, and combinations can be made with respect to the above described embodiments without departing from the spirit and scope of the invention, and that such modifications, alterations, and combinations are to be viewed as being within the ambit of the inventive concept. For example, and referring now to FIG. 8, these embodiments can serve in a feedback loop that facilitates controlled stimulation of muscles during, for example, physical therapy. Pursuant to such a monitor and control application 80, electromyogram signals are processed 81 as before to provide corresponding information regarding the present state of one or more monitored muscles. In a preferred embodiment, this processing occurs at least substantially in real time to thereby afford substantially real time assessment of muscle function response and fatigue levels as pertain to a monitored individual. This process can then effect provision 83 of muscle actuation control signals to cause, for example, a particular muscle (or muscles) to flex. So configured, a person undergoing therapy to regain use of one or more muscles can utilize such selective actuation and monitoring to aid in the rehabilitative process. If desired, these same processes can also be applied in conjunction with a group of muscles to thereby effect controlled actuation of a coordinated group of muscles (such as the muscles for a substantial portion of a leg).

1. An apparatus comprising:
a conformable housing of sufficient size to permit substantially conformal disposition about a human body part;
an electromyogram sensor supported by the conformable housing, such that when the conformable housing is substantially conformally disposed about a human body part the electromyogram sensor can detect muscle activity;
an electromyogram signal processor operably coupled to the electromyogram sensor and being supported by the conformable housing;
a display operably coupled to the electromyogram signal processor and being supported by the conformable housing.

2. The apparatus of claim 1 wherein the conformable housing comprises an elastic member.

3. The apparatus of claim 1 wherein the electromyogram signal processor is substantially disposed within the conformable housing.

4. The apparatus of claim 1 wherein the display comprises a multi-color display.

5. The apparatus of claim 1 wherein the display comprises an alphanumeric display.

6. The apparatus of claim 1 and further comprising at least a second display operably coupled to the electromyogram signal processor and being supported by the conformable housing.

7. The apparatus of claim 1 and further comprising a user input interface.

8. The apparatus of claim 7 wherein the user input interface comprises a switch.

9. The apparatus of claim 1 wherein in the display provides a display that corresponds to a muscle condition parameter.

10. The apparatus of claim 9 wherein the muscle condition parameter corresponds to muscular fatigue.

11. The apparatus of claim 1 and further comprising a portable power source that is supported by the conformable housing and that is operably coupled to the electromyogram signal processor and the electromyogram sensor.

12. The apparatus of claim 11 wherein the portable power source comprises a rechargeable portable power source.

13. The apparatus of claim 12 wherein the rechargeable portable power source comprises a non-contact rechargeable portable power source.

14. The apparatus of claim 1 and further comprising an audible alarm that is supported by the conformable housing and that is operably coupled to the electromyogram signal processor.

15. The apparatus of claim 1 and further comprising a wireless receiver supported by the conformable housing and being operably coupled to the electromyogram signal processor.

16. The apparatus of claim 1 and further comprising a wireless transmitter supported by the conformable housing and being operably coupled to the electromyogram signal processor.

17. The apparatus of claim 1 and further comprising a memory supported by the conformable housing and being operably coupled to the electromyogram signal processor.

18. The apparatus of claim 17 wherein the memory has stored therein at least one of:
strength information;
power information;
a number of muscle flextures;
present rate of muscle flextures;
historical information regarding a rate of muscle flextures;
a number of muscle relaxation events;
present rate of muscle relaxation events;
historical information regarding a rate of muscle relaxation events.

19. A method comprising:
at an electromyogram signal processor supported by a conformable housing that is conformably disposed about a human body part:
receiving electromyogram signals;
processing the electromyogram signals to determine a corresponding muscle condition indicia;
providing a display signal that corresponds to the muscle condition indicia.

20. The method of claim 19 wherein processing the electromyogram signals to determine a corresponding muscle condition indicia comprises processing the electromyogram signals to determine a corresponding muscle condition indicia regarding muscle fatigue.

21. The method of claim 19 wherein receiving electromyogram signals comprises receiving electromyogram signals from at least one electromyogram sensor that is supported by the conformable housing.

22. The method of claim 21 wherein receiving electromyogram signals further comprises also receiving electromyogram signals from at least one electromyogram sensor that is distal to the conformable housing.

23. The method of claim 22 wherein processing the electromyogram signals to determine a corresponding muscle condition indicia comprises processing the electromyogram signals to determine a composite indicia that corresponds to a condition of a plurality of muscles.

24. The method of claim 19 wherein processing the electromyogram signals to determine a corresponding muscle condition indicia comprises utilizing a calibration process to calibrate the processing with respect to at least a first calibration criterion.

25. The method of claim 24 wherein utilizing a calibration process to calibrate the processing with respect to at least a first calibration criterion comprises accessing previously stored calibration information.

26. The method of claim 25 wherein accessing previously stored calibration information comprises accessing at least one of:
locally stored calibration information; and
remotely stored calibration information.

27. The method of claim 24 wherein utilizing a calibration process to calibrate the processing with respect to at least a first calibration criterion includes providing at least one calibration instruction to a user.

28. The method of claim 27 wherein utilizing a calibration process to calibrate the processing with respect to at least a first calibration criterion further comprises receiving electromyogram information in response to providing the at least one calibration instruction to the user.

29. The method of claim 28 wherein receiving electromyogram information in response to providing the at least one calibration instruction to the user comprises receiving electromyogram information within a predetermined period of time of providing the at least one calibration instruction to the user.

30. The method of claim 19 wherein processing the electromyogram signals to determine a corresponding muscle condition indicia comprises using at least a first threshold value that likely corresponds to a user performance parameter.

31. The method of claim 30 and further comprising providing the first threshold value as a function, at least in part, of a calibration process.

32. The method of claim 30 and further comprising providing the first threshold value as a function, at least in part, of past performance by a user.

33. The method of claim 19 and further comprising comparing the corresponding muscle condition indicia with at least one of:
historical muscle condition data;
estimated muscle condition data.

34. A monitor and control method for muscle articulation comprising:
processing at least one electromyogram signal;
providing a substantially real time assessment of muscle function response and fatigue level as a function, at least in part, of the at least one electromyogram signal; and
providing a control signal to control muscle actuation of a local muscle as a function, at least in part, of the substantially real time assessment.

35. The method of claim 34 wherein processing at least one electromyogram signal comprises providing a substantially real time assessment of a muscle function response for a group of muscles.

36. The method of claim 35 wherein providing a control signal to control muscle actuation of a local muscle comprises providing a wireless control signal to a group of devices that control muscle actuation of a coordinated group of muscles.
